(19) United States
(12) Patent Application Publication
(10) Pub. No.: US 2022/0192724 A1
(43) Pub. Date: Jun. 23, 2022

(54) BALLOON PRESSURE AND THRESHOLD CONFIGURATION USING AN ALTIMETER AND PREDEFINED VALUES IN DATABASE

(71) Applicant: Boston Scientific Scimed Inc., Maple Grove, MN (US)

(72) Inventor: El Yacine Alex Smail, Vaudreuil-Dorion, CA (US)

(21) Appl. No.: 17/558,770

(22) Filed: Dec. 22, 2021

Related U.S. Application Data
(60) Provisional application No. 63/129,889, filed on Dec. 23, 2020.

(51) Int. Cl. A61B 18/02

(52) U.S. Cl. CPC A61B 18/02; A61B 2018/0022

(57) ABSTRACT

A cryotherapy system for use in an operating environment, the cryotherapy system comprising a balloon catheter comprising a balloon defining an internal space, and a fluid control system configured to control delivery of a cryogenic fluid to the balloon catheter so as to maintain the internal space at a desired working pressure. The fluid control system comprises a controller configured to receive an external ambient pressure input and to control or calibrate the delivery of the cryogenic fluid based in part on the external ambient pressure input.

### CROSS REFERENCE TO RELATED APPLICATION

This application claims priority to Provisional Application No. 63/129,889, filed Dec. 23, 2020, which is herein incorporated by reference in its entirety.

### TECHNICAL FIELD

The present disclosure relates to medical devices and methods for treating cardiac arrythmias. More specifically, the invention relates to devices and methods for cryogenically ablating cardiac tissue.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few. In particular, catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. The energy delivery component of the system is typically at or near the most distal (i.e. farthest from the user or operator) portion of the catheter, and often at the tip of the catheter.

Various forms of energy can be used to ablate diseased heart tissue. These can include radio frequency (RF), cryogenics, ultrasound and laser energy, to name a few. During a cryoablation procedure, with the aid of a guide wire, the distal tip of the catheter is positioned adjacent to targeted cardiac tissue, at which time energy is delivered to create tissue necrosis, rendering the ablated tissue incapable of conducting electrical signals.

Atrial fibrillation (AF) is a common arrhythmia treated using catheter ablation. One AF treatment strategy involves isolating the pulmonary veins from the left atrial chamber. A particularly useful technique known as catheter balloon cryotherapy or cryoablation can be employed to treat AF. During balloon cryoablation procedures, a balloon on a balloon catheter is positioned within the ostium of the pulmonary vein to be treated, and inflated to intimately contact the surrounding tissue and occlude the pulmonary vein. Fluid is injected into the inflated balloon at cryogenic temperatures to thermally ablate the target tissue.

There is a continuing need for improved systems and methods for controlling balloon pressure for pulmonary vein isolation procedures.

### SUMMARY

Example 1 is a cryotherapy system for use in an operating environment. The cryotherapy system comprises a balloon catheter and a fluid control system. The balloon catheter comprises a balloon defining an internal space. The fluid control system is configured to control delivery of a cryogenic fluid to the balloon catheter so as to maintain the internal space at a desired working pressure. The fluid control system comprises a controller configured to receive an external ambient pressure input and to control or calibrate the delivery of the cryogenic fluid based in part on the external ambient pressure input.

In Example 2, the cryotherapy system of Example 1, wherein the external ambient pressure input is an output from an external sensor.

In Example 3, the cryotherapy system of Example 2, wherein the output from the external sensor is an altitude of the operating environment.

In Example 4, the cryotherapy system of Example 2, wherein the output from the external sensor is a barometric pressure of the operating environment.

In Example 5, the cryotherapy system of Example 2, wherein the external sensor comprises a global positioning system and a database matching the location of the hospital or treatment site to its corresponding altitude. The global positioning system measures a location of a hospital or treatment site. The output from the external sensor is an altitude of the operating environment.

In Example 6, the cryotherapy of Example 1, wherein the fluid control system further comprises a sensor configured to generate the external ambient pressure input based on the operating environment.

In Example 7, the cryotherapy of Example 6, wherein the sensor is configured to sense an altitude of the operating environment.

In Example 8, the cryotherapy of Example 6, wherein the sensor is configured to sense a barometric pressure of the operating environment.

In Example 9, the cryotherapy of Example 6, wherein the sensor is a global positioning system connected to a database matching location of a hospital or treatment site to its corresponding altitude.

In Example 10, the cryotherapy of Example 1, wherein the external ambient pressure input is based on a manual user input regarding the external ambient pressure of the operating environment.

In Example 11, the cryotherapy of Example 10, wherein the manual user input is manually selected from a database matching either location or zip code of a hospital or treatment site to its corresponding altitude.

In Example 12, the cryotherapy of Example 1, further comprises a graphical display system configured to provide dynamic visual data or information to an operator.

In Example 13, the cryotherapy of Example 12, wherein the graphical display system further includes an input field configured to be selectable by the operator to specify the manual input.

In Example 14, the cryotherapy of Examples 1-13, wherein the controller is configured to adjust a balloon working pressure thresholds based on the external ambient pressure input.

In Example 15, the cryotherapy of Examples 1-13, wherein the controller is configured to adjust an injection pressure of the cryogenic fluid based on the external ambient pressure input.

Example 16 is a cryotherapy system for use in an operating environment. The cryotherapy system comprises a balloon catheter and a fluid control system. The balloon catheter comprises a balloon defining an internal space. The fluid control system is configured to control delivery of a cryogenic fluid to the balloon catheter so as to maintain the internal space at a desired working pressure. The fluid control system comprises a controller configured to receive an external ambient pressure input and to control or calibrate the delivery of the cryogenic fluid based in part on the external ambient pressure input.

In Example 17, the cryotherapy of Example 16, wherein the external ambient pressure input is an output from an external sensor.

In Example 18, the cryotherapy of Example 17, wherein the output from the external sensor is an altitude of the operating environment.

In Example 19, the cryotherapy of Example 17, wherein the output from the external sensor is a barometric pressure of the operating environment.

In Example 20, the cryotherapy of Example 17, wherein the external sensor comprises a global positioning system and a database matching the location of the hospital or treatment site to its corresponding altitude. The global positioning system measures a location of a hospital or treatment site. The output from the external sensor is an altitude of the operating environment.

In Example 21, the cryotherapy of Example 16, wherein the external ambient pressure input is based on a manual user input regarding the external ambient pressure of the operating environment.

In Example 22, the cryotherapy of Example 21, wherein the manual user input is manually selected from a database matching either location or zip code of a hospital or treatment site to its corresponding altitude.

In Example 23, the cryotherapy of Example 16, further comprises a graphical display system configured to provide dynamic visual data or information to an operator.

Example 24 is a cryotherapy system for use in an operating environment. The cryotherapy system comprises a balloon catheter and a fluid control system. The balloon catheter comprises a balloon defining an internal space. The fluid control system is configured to control delivery of a cryogenic fluid to the balloon catheter so as to maintain the internal space at a desired working pressure. The fluid control system comprises a sensor configured to generate an external ambient pressure input based on the operating environment and a controller configured to receive an external ambient pressure input and to control or calibrate the delivery of the cryogenic fluid based in part on the external ambient pressure input.

In Example 25, the cryotherapy of Example 24, wherein the sensor is configured to sense an altitude of the operating environment.

In Example 26, the cryotherapy of Example 24, wherein the sensor is configured to sense a barometric pressure of the operating environment.

In Example 27, the cryotherapy of Example 24, wherein the sensor is a global positioning system connected to a database matching location of a hospital or treatment site to its corresponding altitude.

In Example 28, the cryotherapy of Example 24, further comprises a graphical display system configured to provide dynamic visual data or information to an operator.

Example 29 is a method of calibrating a cryotherapy system for use in an operating environment. The cryotherapy system includes a balloon catheter having a balloon defining an internal space, and a fluid control system configured to deliver a cryogenic fluid to the balloon catheter so as to maintain the internal space at a desired working pressure. The method comprises receiving an external ambient pressure input by a controller of the fluid control system and calibrating delivery of the cryogenic fluid based in part on the external ambient pressure input.

In Example 30, the method of Example 29, wherein the external ambient pressure input is an output from an external sensor.

In Example 31, the method of Example 30, wherein the output from the external sensor is an altitude of the operating environment.

In Example 32, the method of Example 30, wherein the output from the external sensor is a barometric pressure of the operating environment.

In Example 33, the method of Example 30, wherein the external sensor comprises a global positioning system and a database matching the location of the hospital or treatment site to its corresponding altitude. The global positioning system measures a location of a hospital or treatment site. The output from the external sensor is an altitude of the operating environment.

In Example 34, the method of Example 29, wherein the external ambient pressure input is based on a manual user input regarding the external ambient pressure of the operating environment.

In Example 35, the method of Example 30, wherein the manual user input is manually selected from a database matching either location or zip code of a hospital or treatment site to its corresponding altitude.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a simplified schematic side-view illustration of an embodiment of a cryotherapy system for use with a patient.

FIG. 2 is a simplified schematic view illustration of a distal end portion of a balloon catheter positioned within a selected anatomical region of a patient, according to an embodiment of the present disclosure.

FIG. 3 is a schematic diagram of a fluid control system according to an embodiment of the present disclosure.

FIG. 4 is a simplified schematic view illustration of a fluid control system according to an embodiment of the present disclosure.

FIG. 5 is an example of a graphical display system illustration according to an alternative embodiment of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

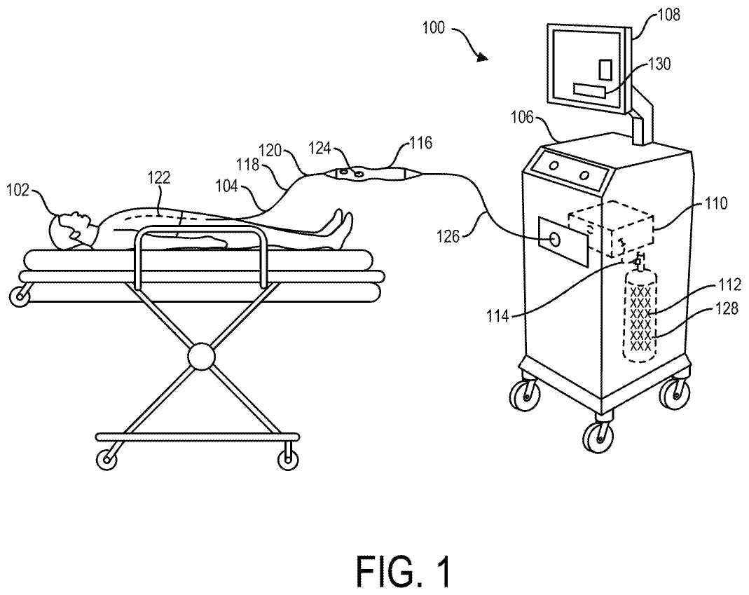

FIG. 1 is a simplified schematic side-view illustration of an embodiment of a cryotherapy system 100 for use with a patient 102, which can be a human being or an animal. Although the design of the cryotherapy system 100 can be varied depending on the particular clinical needs of the patient 102, in the illustrated embodiment, the cryotherapy system 100 can include one or more of a balloon catheter 104 and a fluid control system 106. The fluid control system 106 may include a controller 110 (illustrated in phantom and disposed within the fluid control system 106 in FIG. 1. In some instances, the fluid control system 106 may also include a graphical display system 108.

In the illustrated embodiment, the fluid control system 106 includes a fluid source 112. In the various embodiments, the fluid control system 106 can include various conduits, valves and instrumentation configured to supply and withdraw a fluid to the active elements on the balloon catheter 104 as will be described in greater detail elsewhere herein. In the illustrated embodiment, the fluid source 112 is operably connected to the controller 110 by a fluid delivery line 114 (which may be in the form of a conduit, hose or tubing) configured to transfer fluid contained within the fluid source 112 to components making up the controller 110.

In embodiments, the controller further includes a processor and non-transitory computer-readable memory storing software readable by the processor, whereby the processor can control the operation of the aforementioned valves and other components for controlling the flow of cryogenic fluid to and from the balloon catheter 104.

As further shown, the balloon catheter 104 includes a handle assembly 116 and a shaft 118 having a proximal end portion 120 connected to the handle assembly 116, and a distal end portion 122, shown disposed within the patient 102 in FIG. 1. As will be appreciated, the handle assembly 116 can include various components, such as the control element 124 in FIG. 1, that the user can manipulate to operate the balloon catheter 104. Also, in the particular embodiment illustrated in FIG. 1, an umbilical 126 operatively connects the handle assembly 116 and the active components of the balloon catheter 104 to the fluid control system 106.

In various embodiments, the system 100 may also include additional components or alternative approaches to operatively connect the balloon catheter 104 to the fluid control system 106. That is, the particular means of operatively connecting these elements is not critical the present disclosure, and so any suitable means can be employed.

It is understood that although FIG. 1 illustrates the structures of the cryotherapy system 100 in a particular position, sequence and/or order, these structures can be located in any suitably different position, sequence and/or order than that illustrated in FIG. 1. It is also understood that the cryotherapy system 100 can include fewer or additional components than those specifically illustrated and described herein.

In various embodiments, the controller 110 is configured to monitor and control various processes of the ablation procedures performed with the cryotherapy system 100. More specifically, the controller 110 can monitor and control release and/or retrieval of a cooling fluid 128, e.g., a cryogenic fluid (shown schematically contained within the fluid source 112 in FIG. 1), to the balloon catheter 104, e.g., via fluid injection and fluid exhaust lines (not shown, but which may be disposed within the umbilical 126. The controller 110 can also control various structures that are responsible for maintaining and/or adjusting a flow rate and/or pressure of the cryogenic fluid 128 that is released to the balloon catheter 104 during the cryoablation procedure. In such embodiments, the cryotherapy system 100 delivers ablative energy in the form of cryogenic fluid 128 to cardiac tissue of the patient 102 to create tissue necrosis, rendering the ablated tissue incapable of conducting electrical signals. Additionally, in various embodiments, the controller 110 can control activation and/or deactivation of one or more other processes of the balloon catheter 104.

Further, or in the alternative, the controller 110 can receive data and/or other information (hereinafter sometimes referred to as “sensor output”) from various structures within the cryotherapy system 100. In some embodiments, the controller 110 can receive, monitor, assimilate and/or integrate the sensor output, and/or any other data or information received from any structure within the cryotherapy system 100 in order to control the operation of the balloon catheter 104. For example, the controller may be configured to receive an external ambient pressure input and to control or calibrate the delivery of the cryogenic fluid 128 based in part on the external ambient pressure input. As will be further discussed below, the external ambient pressure input may be based on a sensor or a manual user input. As provided herein, in various embodiments, the controller 110 can initiate and/or terminate the flow of cryogenic fluid 128 to the balloon catheter 104 based on the sensor output.

As shown in FIG. 1, in certain embodiments, the controller 110 can be positioned substantially within the fluid control system 106. Alternatively, at least a portion of the controller 110 can be positioned in one or more other locations within the cryotherapy system 100, e.g., within the handle assembly 116.

The fluid source 112 contains the cryogenic fluid 128, which is delivered to and from the balloon catheter 104 with or without input from the controller 110 during a cryoablation procedure. Once the ablation procedure has initiated, the cryogenic fluid 128 can be delivered and the resulting gas, after a phase change, can be retrieved from the balloon catheter 104, and can either be vented or otherwise discarded as exhaust. Additionally, the type of cryogenic fluid 128 that is used during the cryoablation procedure can vary. In one non-exclusive embodiment, the cryogenic fluid 128 can include liquid nitrous oxide. However, any other suitable cryogenic fluid 128 can be used. For example, in one non-exclusive alternative embodiment, the cryogenic fluid 128 can include liquid nitrogen.

The design of the balloon catheter 104 can be varied to suit the specific design requirements of the cryotherapy system 100. As shown, the balloon catheter 104 is inserted into the body of the patient 102 during the cryoablation procedure. The handle assembly 116 can be handled and used by the operator to operate, position and control the balloon catheter 104. The design and specific features of the handle assembly 116 can vary to suit the design requirements of the cryotherapy system 100. In the embodiment illustrated in FIG. 1, the handle assembly 116 is separate from, but in electrical and/or fluid communication with the controller 110, the fluid source 112, and the graphical display system 108. In some embodiments, the handle assembly 116 can integrate and/or include at least a portion of the controller 110 within an interior of the handle assembly 116. It is understood that the handle assembly 116 can include fewer or additional components than those specifically illustrated and described herein. Additionally, in certain embodiments, the handle assembly 116 can include circuitry (not shown in FIG. 1) that can include at least a portion of the controller 110. Alternatively, the circuitry can transmit electrical signals such as the sensor output, or otherwise provide data to the controller 110 as described herein. In one embodiment, the circuitry can include a printed circuit board having one or more integrated circuits, or any other suitable circuitry.

Still further, in certain embodiments, the handle assembly 116 can be used by the operator to initiate and/or terminate the cryoablation process, e.g., to start the flow of the cryogenic fluid 128 to the balloon catheter 104 in order to ablate certain targeted heart tissue of the patient 102.

In the embodiment illustrated in FIG. 1, the fluid control system 106 includes at least a portion of the controller 110, the fluid source 112, and the graphical display system 108. However, in alternative embodiments, the fluid control system 106 can contain additional structures not shown or described herein. Still alternatively, the fluid control system 106 may not include various structures that are illustrated within the fluid control system 106 in FIG. 1. For example, in certain non-exclusive alternative embodiments, the fluid control system 106 does not include the graphical display system 108.

During cryoablation procedures, the balloon catheter 104 and the fluid control system 106 must be mechanically connected to allow the flow of cryogenic fluid 128 from the fluid control system 106 to the balloon catheter 104 and back to the fluid control system 106. Generally, during the application of ablative energy, the cryogenic fluid 128 flows in a liquid phase to the balloon catheter 104. The cryogenic fluid 128 then undergoes a phase change and returns to the fluid control system 106 as exhaust in a gaseous phase.

In various embodiments, the graphical display system 108 is electrically connected to the controller 110. Additionally, the graphical display system 108 provides the operator of the cryotherapy system 100 with information that can be used before, during and after the cryoablation procedure. For example, the graphical display system 108 can provide the operator with information based on the sensor output, and any other relevant information that can be used before, during and after the cryoablation procedure. The specifics of the graphical display system 108 can vary depending upon the design requirements of the cryotherapy system 100, or the specific needs, specifications and/or desires of the operator.

In one embodiment, the graphical display system 108 can provide static visual data and/or information to the operator via various frames or other representations (depicted as element 130 in FIG. 1). In addition, or in the alternative, the graphical display system 108 can provide dynamic visual data and/or information to the operator, such as video data or any other data that changes over time, e.g., during an ablation procedure. Further, in various embodiments, the graphical display system 108 can include one or more colors, different sizes, varying brightness, etc., that may act as alerts to the operator. Additionally, or in the alternative, the graphical display system 108 can provide audio data or information to the operator.

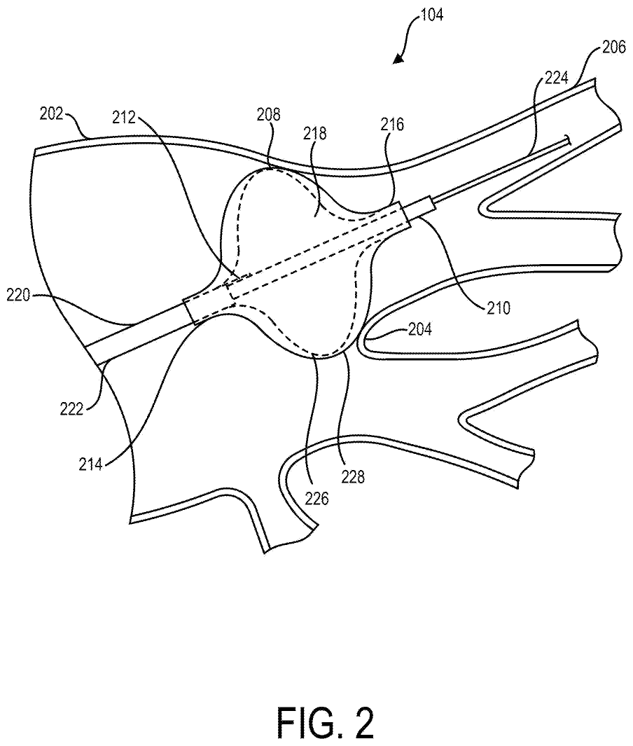

FIG. 2 is a schematic view illustration of the distal end portion 122 of the balloon catheter 104 positioned within a selected anatomical region of the patient 102, according to an embodiment of the present disclosure. In this case, a left atrium 202 is adjacent to an ostium 204 of a pulmonary vein 206, such as when the cryotherapy system 100 is used in a pulmonary vein isolation (“PVI”) procedure to terminate an atrial fibrillation. In the illustrated embodiment, the balloon catheter 104 includes a balloon 208, a guidewire lumen 210 and an injection tube 212. As shown, the balloon 208 has a proximal end 214 and an opposite distal end 216, and defines an internal space 218 that creates a cryo-chamber during a cryoablation procedure. In the illustrated embodiment, the proximal end 214 of the balloon 208 is attached to the distal end portion 220 of a shaft 222, and the distal end 216 of the balloon 208 is attached to the guidewire lumen 210 near the distal end thereof. In the illustrated embodiment, the injection tube 212 is disposed within and extends from the shaft 222, and terminates within and is open to the internal space 218. The injection tube 212 is operable to deliver the cryogenic fluid 128 to the internal space 218.

Although not shown in FIG. 2, the balloon catheter 104 also includes an exhaust lumen within the shaft 222 and open to the internal space 218. The exhaust lumen is operable to facilitate evacuation of the cryogenic fluid 128 from the internal space 218, and also to facilitate inflation of the balloon 208 as will be explained in further detail herein.

In various embodiments, the guidewire lumen 210 may be slidable relative to the shaft 222 to facilitate expansion and subsequent collapse of the balloon 208 in use. In certain embodiments, the balloon 208 is expandable. In other embodiments, the balloon 208 is inflatable. However, the particular construction of the balloon 208 and guidewire lumen 210 is not critical to the present disclosure, and so other configurations may be used within the scope of the various embodiments.

For illustration purposes, an instrument 224 is shown extending through and beyond the guidewire lumen and into the pulmonary vein 206. As the skilled artisan will appreciate, the instrument 224 may be a guidewire, mapping wire or catheter, anchoring wire, or other medical device useful to facilitate the particular cryotherapy procedure. However, the use of the instrument 224 is optional and is not critical to the embodiments disclosed herein.

In the embodiment of FIG. 2, the balloon 208 is a dual-balloon construction including an inner balloon 226 and an outer balloon 228. The inner and outer balloons 226, 228 are configured such that the inner balloon 226 receives the cryogenic fluid 128 (illustrated in FIG. 1), and the outer balloon 228 surrounds the inner balloon 226. The outer balloon 228 acts as part of a safety system to capture the cryogenic fluid 128 in the event of a leak from the inner balloon 226. It is understood that the balloon catheter 104 can include other structures as well. However, for the sake of clarity, these other structures have been omitted from the figures. Additionally, it is further appreciated that in some alternative embodiments, the balloon catheter 104 includes only a single balloon.

In the embodiment illustrated in FIG. 2, the balloon catheter 104 is positioned within the left atrium 202 of the patient 102. The guidewire 224 and guidewire lumen 210 are inserted into a pulmonary vein 206 of the patient 102, and the catheter shaft 118 and the inner and outer balloons 226, 228 are moved along the guidewire 224 and/or the guidewire lumen 210 to be positioned near an ostium 204 of the pulmonary vein 206.

During use, the inner balloon 226 can be partially or fully inflated so that at least a portion of the inner balloon 226 expands against at least a portion of the outer balloon 228. Once the inner balloon 226 is sufficiently inflated, an outer surface of the outer balloon 228 can then be positioned to abut and/or substantially form a seal with the ostium 204 of the pulmonary vein 206 to betreated.

The inner balloon 226 and the outer balloon 228 can be formed from any suitable materials. For example, in some embodiments, the inner balloon 226 can be formed from a sturdy material to better inhibit leaks of the cryogenic fluid 128 that is received therein, and the outer balloon 228 can be made from a relatively compliant material to ensure better contact and positioning between the outer balloon 228 and the pulmonary vein 206.

During balloon cryoablation procedures, prior to delivering the cryoablative energy, the operator can inflate the balloon using the cryogenic fluid 128 at a relatively high temperature (i.e., well above the temperature sufficient to ablate the target tissue). In this way, the operator can ensure sufficient balloon-tissue contact and vein occlusion before starting an ablation to increase probability of vein isolation. It is desirable to maintain relatively close control over the inflation pressure during the cryoablation procedure. For example, a drop in the inflation pressure can result in partial deflation of the balloon 208 and consequent or diminishment of balloon tissue contact and vessel occlusion. At the same time, over-inflation of the balloon 208 can also tend to push the balloon 208 out of the vein, which can also result in a loss or diminishment of vein occlusion.

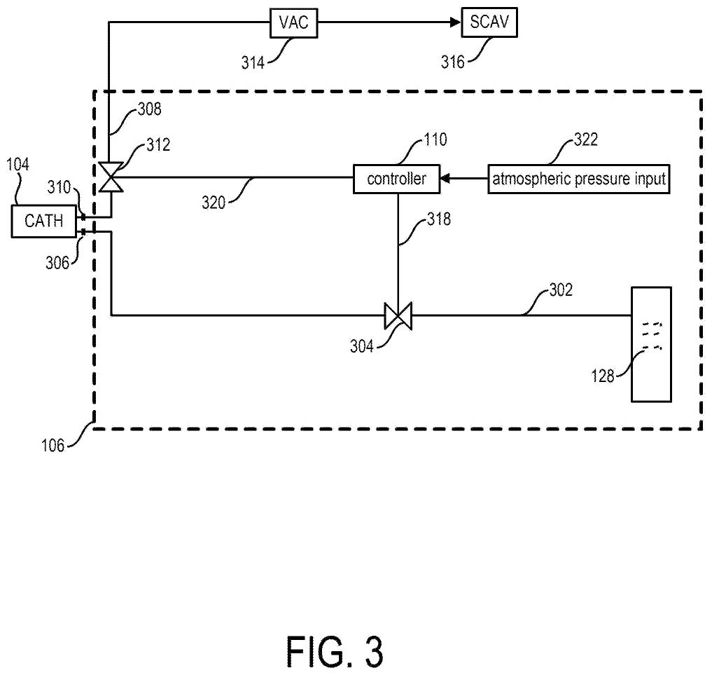

FIG. 3 is a schematic diagram of a fluid control system 106 according to an embodiment of the present disclosure. In the illustrated embodiment, the fluid control system 106 includes the fluid source 128, which is operatively and fluidly coupled to the balloon catheter 104 via an injection line 302. As further shown, the injection line 302 includes a supply valve 304 and an injection port 306 on the balloon catheter 104.

Additionally, FIG. 3 illustrates an exhaust line 308 fluidly coupled to a return port 310 on the balloon catheter 104. As shown, the exhaust line 308 includes an exhaust valve 312 and a vacuum pump 314, and is operatively connected to a scavenging system 316 (e.g., via a wall-mounted port in the electrophysiology lab). The exhaust line 308 is operable to evacuate the cryogenic fluid 128 from the balloon catheter 104 and thereby effect deflation of the balloon 208 as per the needs of the operator, as is well known in the art.

As shown, the controller 110 is operably connected to the supply valve 304 via an injection control cable 318, and is operably connected to the exhaust valve 312 via an exhaust control cable 320. In certain embodiments, the injection control cable 318 and the exhaust control cable 320 may be disposed within the umbilical 126, as mentioned above.

In one embodiment, the controller 110 is configured to receive an external ambient pressure input 322. As will be further discussed below, in some instances, the external ambient pressure input 322 may be based on a sensor that measures the ambient pressure or altitude of the operating environment. In other instances, the external ambient pressure input 322 may be based on a global positioning system that is connected to a database matching location of a hospital or treatment site to its corresponding altitude and, consequently, a representative atmospheric pressure of the geographical location in which the operating environment resides. In yet other instances, the external ambient pressure input 322 may be based on a manual user input regarding the external ambient pressure of the operating environment, or selecting from a database matching location (e.g., geographic coordinates, zip code, and the like) of a hospital or treatment site to its corresponding altitude.

In embodiments, various operational aspects of the cryotherapy system 100 can be affected by the external atmospheric or ambient pressure of the operating environment in which the cryotherapy system 100 is used. Typically, however, the cryotherapy system 100 and its various components, including the balloon catheter 104, the fluid control system 106 and the various sensors (e.g., balloon pressure sensors) used for controlling delivery and/or exhaust of the cryogenic fluid 128 to and from the balloon 108, are assembled and calibrated at a different location than the operating environment in which the cryotherapy system 100 is used for patient treatment. In embodiments, in response to the external ambient pressure input, the controller 110 automatically compensates balloon pressure control parameters and/or calibration thereof in relation to the external ambient pressure input 322.

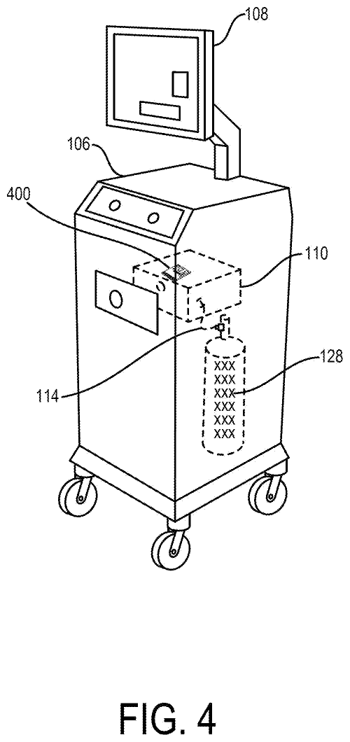

FIG. 4 is a simplified schematic view illustration of the fluid control system 106 according to an embodiment of the present disclosure. As shown, the controller 110 disposed within the fluid control system 106 further includes a sensor 400. The controller 110 is configured to receive an output from the sensor 400 that is configured to generate an output that corresponds to the external ambient pressure input. In embodiments, the sensor 400 is configured to directly sense the atmospheric pressure of the operating environment and generate a sensor output that represents the external ambient pressure input, which is received by the controller 110, and selected operating and/or calibration parameters are thereby adjusted to compensate for differences between the actual operating environment atmospheric pressure and the manufacturing site conditions. In embodiments, the sensor 400 is an electronic barometric pressure sensor.

In other embodiments, the sensor 400 senses a different operating environment condition (i.e., other than atmospheric pressure), and the output of the sensor 400 is received by the controller 110. In such embodiments, the controller 110 also includes or is able to access a relational database that associates the sensed condition with representative atmospheric pressure values. In one such embodiment, the sensor 400 is an electronic altitude sensor and the relational database includes associations between altitude and atmospheric pressure. In other embodiments, the sensor 400 is a global positioning system connected to a database matching location of a hospital or treatment site to its corresponding altitude, or alternatively, directly associates the detected location with atmospheric pressure values. In embodiments, the sensor 400 can include both altitude and location sensing capabilities.

Based on the external ambient pressure input from the sensor, the controller 110 compensates selected parameters to control the delivery of the cryogenic fluid 128 in relation to the external ambient pressure input 322. Exemplary parameters include low and high pressure set points, low and high flow set points, low and high target injection pressure, maximum and minimum acceptable pressure limits for the working balloon 208, maximum exhaust pressure under vacuum, and the like.

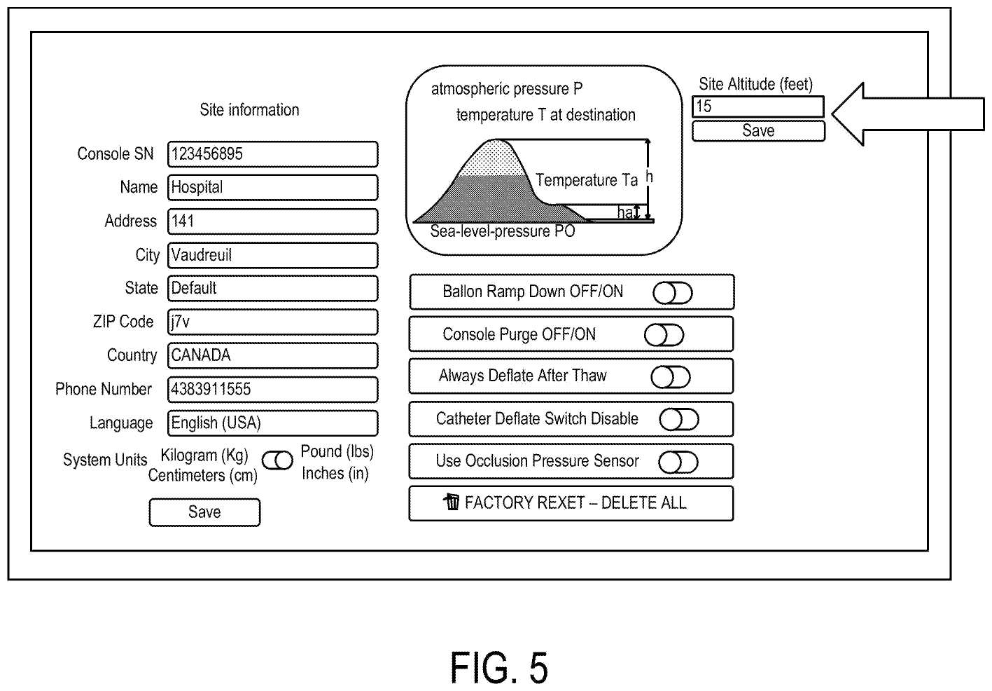

In other embodiments, the external ambient pressure input 322 is in the form of a manual user input. FIG. 5 depicts an exemplary graphical display system 108 illustration according to an alternative embodiment of the present disclosure. The graphical display system 108 is configured to provide dynamic visual data and/or information to an operator. As shown, the graphical display system 108 includes a manual external ambient pressure input 322 whereby the user of the cryotherapy system 100 can manually specify information indicative of the atmospheric pressure of the operating environment to be used by the controller 110 as explained above. In one embodiment, the manual external ambient pressure input 322 is a manually-obtained reading of the barometric pressure of the operating environment (e.g., from a manual reading of a barometer). In one embodiment, the manual external ambient pressure input 322 is based on a manual user input that reflects the altitude of the operating environment (e.g., from a manual reading of an altimeter), which can be associated to a representative barometric pressure value for use by the controller 110. In yet another embodiment, the manual external ambient pressure input 322 can be the hospital or medical institution name and/or location (e.g., based on a postal code, city name, and the like), and the controller 110 can access a database matching the input value(s) to a corresponding atmospheric pressure value as described above.

In embodiments, the manual external ambient pressure input 322 can take on any number of forms. For example, in the illustrated embodiment, the user can input (via, e.g., a keyboard) the aforementioned atmospheric pressure, altitude or location information. In embodiments, the graphical display system 108 can include a pull-down menu through which the user can select the desired value(s). The skilled artisan will readily recognize that still other implementations may be employed within the scope of the present disclosure based on the foregoing.

It is understood that although a number of different embodiments of the cryotherapy system 100 have been illustrated and described herein, one or more features of any one embodiment can be combined with one or more features of one or more of the other embodiments, provided that such combination satisfies the intent of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A cryotherapy system for use in an operating environment, the cryotherapy system comprising:
a balloon catheter comprising a balloon defining an internal space; and
a fluid control system configured to control delivery of a cryogenic fluid to the balloon catheter so as to maintain the internal space at a desired working pressure;
wherein the fluid control system comprises a controller configured to receive an external ambient pressure input and to control or calibrate the delivery of the cryogenic fluid based in part on the external ambient pressure input.

2. The cryotherapy system of claim 1, wherein the external ambient pressure input is an output from an external sensor.

3. The cryotherapy system of claim 2, wherein the output from the external sensor is an altitude of the operating environment.

4. The cryotherapy system of claim 2, wherein the output from the external sensor is a barometric pressure of the operating environment.

5. The cryotherapy system of claim 2, wherein the external sensor comprises:
a global positioning system measuring a location of a hospital or treatment site; and
a database matching the location of the hospital or treatment site to its corresponding altitude;
wherein the output from the external sensor is an altitude of the operating environment.

6. The cryotherapy system of claim 1, wherein the external ambient pressure input is based on a manual user input regarding the external ambient pressure of the operating environment.

7. The cryotherapy system of claim 6, wherein the manual user input is manually selected from a database matching either location or zip code of a hospital or treatment site to its corresponding altitude.

8. The fluid control system of claim 1, further comprising a graphical display system configured to provide dynamic visual data or information to an operator.

9. A cryotherapy system for use in an operating environment, the cryotherapy system comprising:
a balloon catheter comprising a balloon defining an internal space; and
a fluid control system configured to control delivery of a cryogenic fluid to the balloon catheter so as to maintain the internal space at a desired working pressure, wherein the fluid control system comprises:
a sensor configured to generate an external ambient pressure input based on the operating environment; and
a controller configured to receive the external ambient pressure input and to control or calibrate the delivery of the cryogenic fluid based in part on the external ambient pressure input.

10. The cryotherapy system of claim 9, wherein the sensor is configured to sense an altitude of the operating environment.

11. The cryotherapy system of claim 9, wherein the sensor is configured to sense a barometric pressure of the operating environment.

12. The cryotherapy system of claim 9, wherein the sensor is a global positioning system connected to a database matching location of a hospital or treatment site to its corresponding altitude.

13. The fluid control system of claim 9, further comprising a graphical display system configured to provide dynamic visual data or information to an operator.

14. A method of calibrating a cryotherapy system for use in an operating environment, the cryotherapy system including a balloon catheter having a balloon defining an internal space, and a fluid control system configured to deliver a cryogenic fluid to the balloon catheter so as to maintain the internal space at a desired working pressure, the method comprising:
receiving an external ambient pressure input by a controller of the fluid control system; and
calibrating delivery of the cryogenic fluid based in part on the external ambient pressure input.

15. The method of claim 14, wherein the external ambient pressure input is an output from an external sensor.

16. The method of claim 15, wherein the output from the external sensor is an altitude of the operating environment.

17. The method of claim 15, wherein the output from the external sensor is a barometric pressure of the operating environment.

18. The method of claim 15, wherein the external sensor comprises:
a global positioning system measuring a location of a hospital or treatment site; and
a database matching the location of the hospital or treatment site to its corresponding altitude;
wherein the output from the external sensor is an altitude of the operating environment.

19. The method of claim 14, wherein the external ambient pressure input is based on a manual user input regarding the external ambient pressure of the operating environment.

20. The cryotherapy system of claim 19, wherein the manual user input is manually selected from a database matching either location or zip code of a hospital or treatment site to its corresponding altitude.
